(19) United States
(12) Patent Application Publication
(10) Pub. No.: US 2009/0136923 A1
(43) Pub. Date: May 28, 2009

(54) PRIMER COMPOSITION FOR DETECTING MYCOBACTERIUM SP. AND THE DETECTION METHOD

(75) Inventors: Eun Park, Gyeonggi-do (JP); Moo-Joo Lee, Seoul (KR); Kyung-Ryul Lee, Seoul (KR)

Correspondence Address: FOLEY & LARDNER LLP, 777 EAST WISCONSIN AVENUE, MILWAUKEE, WI, 53202-5306, US

(73) Assignee: Biocore Co., Ltd

(21) Appl. No.: 11/722,160

(22) PCT Filed: Dec. 20, 2005
(86) PCT No.: PCT/KR05/04401

§ 371 Date: Jun. 19, 2007

(30) Foreign Application Priority Data
Dec. 20, 2004 (KR) 10-2004-0108738

(51) Int. Cl. C12Q 1/68; C07H 21/04

(52) U.S. Cl. 435/6; 536/24.33

(57) ABSTRACT

Disclosed are a primer composition including two pairs of primers targeting one gene (mpb64) concerning Mycobacterium sp., and a pair of primers targeting a human-derived gene (HLA-DR); and a method for detecting Mycobacterium sp. using a multiplex one-tube nested PCR method in which their primers are used to amplify two genes in one tube at the same time. The method of the present invention may provide a useful guide post capable of clinically diagnosing Mycobacterium sp. in a specimen in a more specific, rapid and convenient manner.

### TECHNICAL FIELD

The present invention relates to a primer composition including two pairs of primers targeting one gene (mpb64) concerning Mycobacterium sp., and a pair of primers targeting a human-derived gene (HLA-DR); and a method for detecting Mycobacterium sp. using a multiplex one-tube nested PCR method in which their primers are used to amplify two genes in one tube at the same time. More particularly, the present invention relates to a primer composition including two pairs of primers targeting a mpb64 gene, which are more specific to Mycobacterium sp., in order to solve a false-positive problem of an IS6110 gene mainly used for detecting human-type and bovine-type tuberculosis bacilli, and a pair of primers targeting a human-derived gene (HLA-DR), which are used for confirming whether or not the reaction may be successfully accomplished, in order to solve a false-negative problem of the IS6110 gene; and a method for amplifying two genes in one tube at the same time using the three pairs of the primers, and diagnosing the amplified product.

### BACKGROUND ART

Members of the Mycobacteria genus contain a number of pathogens that infect humans and animals to develop symptoms of tuberculosis, leprosy, etc. and there have been currently approximately 70 known species in the art. The members of the Mycobacteria genus include the human-type tuberculosis bacilli (Mycobacterium tuberculosis) known as the most important pathogens which infect humans to develop symptoms of tuberculosis, the bovine-type tuberculosis bacilli (Mycobacterium bovis) which occur at a rare frequency, and also Mycobacterium leprae which infects humans to develop symptoms of leprosy (Shinnick T, M, etc., Eur. J. Clin. Microbiol. Infect Dis. 1994; 13(11): 884-901). Tuberculosis caused by infections of the human-type tuberculosis bacilli (Mycobacterium tuberculosis) and the rarely frequent bovine-type tuberculosis bacilli are ones of the diseases which occur in underdeveloped countries and often have their outbreaks in Korea. According to a statistical survey by the Korean National Institute of Health, it was known that 1.03 out of 100 Koreans have suffered from the tuberculosis (1995), and 7.1 out of a hundred thousand Koreans have died from pulmonary tuberculosis (1997).

Recently, it was also reported that it has been difficult to treat the tuberculosis as the pathogens increasingly possess a multiple-drug resistance to the therapeutic agents for treatment of the tuberculosis (Kam K. M. etc., Clin. Infect. Dis. 2002; 34(3):324-9; Barnes, P. etc., J. Med, 1991; 324: 1644-1650).

Accordingly, there has been a necessary demand to diagnose Mycobacterium sp. at an early stage for a suitable treatment.

The general methods for diagnosing Mycobacterium sp. include patient's clinical symptoms, a tuberculin test, X-ray photography, an inspection of Mycobacterium sp., etc. The tuberculin test, known as one of the most simple methods, may be simply conducted, but proven to be negative when patients suffer from a severe tuberculosis, the measles, and anergy owing to immune suppressions.

Also, a smearing/staining method has been generally used in general laboratories to measure acid fastness by Ziehl-Neelsen staining, but the results may be obtained simply and rapidly, but sensitivity may be reduced according to the said method.

As the culture method having a high sensitivity, there has been a method in which a sample of Mycobacterium sp. is cultured at 37° C. for about 4 to 8 weeks under a CO₂ partial pressure of 5 to 10%, but this method has a disadvantage that it is not suitable for treating Mycobacterium sp. due to a very long period of inspection.

Recently, it is possible to precisely and rapidly detect Mycobacterium sp. from clinical specimens only in a day using a polymerase chain reaction (PCR) and a multiplex one-tube nested PCR method, and this method has been widely used since it was reported that the method had the sensitivity and specificity of at least 95% (Wilson S. M. etc; J. Clin. Microbiol. 1993; 31(4): 776-782 and Noordhoek G. T. etc., J. Clin. Microbiol. 1994; 32(2): 277-84).

Various genes and their fractions have been generally used in the PCR method for diagnosing the tuberculosis, that is, IS6110 and 16S rRNA have been most widely used in general (Noordhoek G. T. etc., J. Clin. Microbiol. 1996; 34(10): 2522-2525), and also hsp65, mtp40 and 32-KDa have been also used in the art (Brunello F. etc., J. Clin. Microbiol. 2001; 39(8): 2799-2806 and Herrera E. A. etc., J. Clin. Microbiol. 1996; 34(5): 1108-1113). However, the clinical tests have been reported that each of the genes displayed false positive or false negative results (IS6110 -B. W. Lee etc., Journal of the Neurological Sciences 1994; 123:173-179, 16SrRNA-Donald L. Jungkind etc., J. Clin. Microbiol. 1995; 33(10); 2582-2586, hsp65 - B. W. Lee etc., Journal of the Neurological Sciences 1994; 123:173-179, mtp40 - Marchetti G. etc., J Clin Microbiol. 1998; 36(6): 1512-1517), and therefore it had a disadvantage that it was very difficult to clinically diagnose the tuberculosis.

### DISCLOSURE OF INVENTION

Technical Problem

Accordingly, the present invention is designed to solve the problems of the prior art, and therefore it is an object of the present invention to provide a primer composition for a multiplex one-tube nested PCR capable of amplifying specific genetic regions from the Mycobacteria group and human-derived gene-specific genes (mpb64, HLA-DR) with an excellent sensitivity at the same time.

It is another of the present invention to provide a method capable of detecting and diagnosing the Mycobacteria group at the same time by means of a multiplex one-tube nested PCR method using the primer composition.

Technical Solution

In order to accomplish the above object, the present invention provides a primer composition for detecting Mycobacterium sp., including at least two primers selected from the group consisting of primers set forth in SEQ ID NO: 1 to SEQ ID NO: 6. In the present invention, the composition preferably includes at least one primer selected from the group consisting of SEQ ID NO: 1 to SEQ ID NO: 4, and a primer selected from the group consisting of SEQ ID NO: 5 and SEQ ID NO: 6. In the present invention, the composition more preferably includes all of the primers set forth in SEQ ID NO: 1 to SEQ ID NO: 6.

Also, the present invention provides a kit for detecting Mycobacterium sp., including a container containing the primer composition of the present invention.

Also, the present invention provides a method for detecting Mycobacterium sp., including steps of a) extracting DNA from a sample; b) mixing the primer compositions as defined in claim 1 or 2 with the extracted DNA in the same container; c) PCR-amplifying the DNA in the primer mixture; and d) verifying the amplified PCR product.

In the present invention, the sample is preferably selected from the group consisting of blood, sputum, saliva, urine, cerebrospinal fluid, pleural fluids, pleural biopsies or bronchial washing fluid, and most preferred is sputum.

### BRIEF DESCRIPTION OF THE DRAWINGS

These and other features, aspects, and advantages of preferred embodiments of the present invention will be more fully described in the following detailed description, taken accompanying drawings. In the drawings:

FIG. 1 is an electrophoretic gel image showing a result that a HLA-DR gene and a mpb64 gene are amplified in one tube at the same time using a multiplex one-tube nested PCR method. In FIG. 1, “M” represents a size marker used herein, “P” represents a positive control, “N” represents a negative control, Lane 1 represents a TB negative specimen, and Lanes 2 and 3 represent TB positive specimens.

FIG. 2 is an electrophoretic gel image showing a result of a sample test using a Mpb64 PCR kit of the present invention.

FIG. 3 is an electrophoretic gel image showing a result of a sample test using a conventional IS6110 PCR kit. In FIGS. 2 and 3, “M” represents a PhiX174 Hae III marker, “P” represents a positive control, “N”represents a negative control, and Lanes 1 to 44 represent clinical samples.

FIG. 4 is a table showing a test result comparing the Mpb64 PCR kit of the present invention to the conventional IS6110 PCR kit.

### BEST MODE

Hereinafter, preferred embodiments of the present invention will be described in detail referring to the accompanying drawings.

The present inventors have developed a multiplex one-tube nested PCR method capable of detecting Mycobacterium sp. in a single reaction by selecting a mpb64 gene and a human-derived gene (HLA-DR), wherein the mpb64 gene is specifically present in both of the human-type and bovine-type tuberculosis bacilli in order to detect the human-type tuberculosis bacilli (Mycobacterium tuberculosis) and the bovine-type tuberculosis bacilli (Mycobacterium bovis) at the same time without any of false-positive and false-negative possibilities, and the human-derived gene (HLA-DR) may be used for confirming whether or not the reaction may be successfully accomplished. In the present invention, three pairs of primers which have different characteristics respectively were particularly selected by analyzing two genes which have different characteristics respectively so that the two genes can be amplified in one tube using the three pairs of primers, and therefore diagnostic sensitivity for detecting Mycobacterium sp. was significantly improved in one embodiment of the multiplex one-tube nested PCR method. Accordingly, the present inventors have ardently attempted to develop an easy, sensitive and simple method for detecting and diagnosing both of the human-type and bovine-type tuberculosis bacilli at the same time. As a result, the present inventors have constructed two pairs of novel primers (a target gene: mpb64 gene) specific to the M. tuberculosis group (M. tuberculosis and M. bovis) and a pair of novel primers (a target gene: HLA-DR) specific to the human-derived gene, and then they have found that a M. tuberculosis group can be detected and diagnosed with the excellent sensitivity and specificity by carrying out a multiplex one-tube nested PCR method in which three pairs of the genes are amplified in one tube at the same time using template DNA in the specimen and the constructed primers, and therefore the present invention has been completed.

The present invention relates to a primer composition including two pairs of primers targeting one gene (mpb64) concerning Mycobacterium sp., and a pair of primers targeting a human-derived gene (HLA-DR) used for confirming whether or not the reaction may be successfully accomplished; and a method for detecting Mycobacterium sp. using a multiplex one-tube nested PCR method in which their primers are used to amplify two genes in one tube at the same time. More particularly, the present invention relates to a primer composition including two pairs of primers targeting a mpb64 gene, which are more specific to Mycobacterium sp., in order to solve a false-positive problem of an IS6110 gene mainly used for detecting human-type and bovine-type tuberculosis bacilli, and a pair of primers targeting a human-derived gene (HLA-DR), which are used for confirming whether or not the reaction may be successfully accomplished, in order to solve a false-negative problem of the IS6110 gene; and a method for amplifying two genes in one tube at the same time using the three pairs of the primers, and diagnosing the amplified product. Accordingly, the present invention may provide a useful guide post capable of clinically diagnosing Mycobacterium sp. in a specimen in a more specific, rapid and convenient manner.

In the present invention, the multiplex one-tube nested PCR method was carried out in one tube at the same time by using two pairs of primers designed to target a gene region (mpb64) specific to both of the M. tuberculosis group and DNA extracted from the clinical specimen, and a pair of primers designed to target a human-derived gene (HLA-DR).

It has been known that the mpb64 gene is an immunogenic protein gene specifically present in the human-type tuberculosis bacilli (Mycobacterium tuberculosis) and the bovine-type tuberculosis bacilli (Mycobacterium bovis), and one copy of the mpb64 gene is present in the M. tuberculosis group, and it was confirmed that the gene has excellent sensitivity and specificity (B. W. Lee etc., Journal of the Neurological Sciences 1994; 123:173-179).

Also, the HLA-DR gene is one of genes involving the immune system in human bodies and suitably used as an internal control factor (Oytip Na Thalang, etc, Southeast Asian J Trop Med Public Health 1996; 27(1); 169-171).

The multiplex one-tube nested PCR method was carried out using the three pairs of the primers of the present invention. As a result, it was confirmed that the M. tuberculosis group might be detected and diagnosed in the specimens, as well as the false-negative possibility may be excluded.

### MODE FOR INVENTION

Hereinafter, the present invention will be described in detail referring to non-limiting embodiments. However, it should be understood that the detailed description and specific examples, while indicating preferred embodiments of the invention, are given by way of illustration only, as be apparent to those skilled in the art from this detailed description.

### EXAMPLE

### Example 1

### Construction of Primers for Multiplex PCR

It was confirmed that mpb64-specific primers used herein were primer sequences that can amplify only the M. tuberculosis group (M. tuberculosis and M. bovis) by analyzing the DNA sequence, deposited with accession No. AE000516 in GenBank (www.ncbi.nlm.nih.gov) managed by National Center for Biotechnology Information (NCBI) of U.S. National Institutes of Health (NIH), using a DNAsis program from the company Hitachi Software, sequencing the DNA sequence, and then analyzing the DNA sequence again with BLAST (www.ncbi.nlm.nih.gov/BLAST/).

Also, it was confirmed that HLA-DR-specific primers used herein were primer sequences that canamplify only the HLA-DR gene by analyzing the DNA sequence, with accession No. AY305859 deposited in GenBank, using the DNAsis program, sequencing the DNA sequence, and then analyzing the DNA sequence again with BLAST.

DNA sequences of the constructed primers, and sizes of genes amplified by the multiplex one-tube nested PCR method using the constructed primers are listed in the following Table 1.

TABLE 1

| | | | |
|---|---|---|---|
| Target | | | PCR-amplified |
| Gene | Primer | DNA sequence (5′→3′) | Size |

| | | | | |
|---|---|---|---|---|
| MpbG4 | Primary | Sense | ccc agt tac tac ccc gac cag aag tc (SEQ ID NO: 1) | 276 bp |
| Mpb64 | Secondary | Antisense | cca cag cgt gtc ata ggt gat tgg ctt gc | |
| | | | (SEQ ID NO: 2) | |
| Mpb64 | Secondary | Sense | agt cgc tgg aaa att aca tcg (SEQ ID NO: 3) | 170 bp |
| Mpb64 | | Antisense | ggc gtt ctg gta gac ctt (SEQ ID NO: 4) | |
| HLA-DR | | Sense | tcc cca cag cac gtt tct tg (SEQ ID NO: 5) | 276 bp |
| HLA-DR | | Antisense | ccg ctg cac tgt gaa gct ct (SEQ ID NO: 6) | |

### Example 2

### Synthesis of the Primers

The primers analyzed in Example 1 were synthesized by the method such as “Synthesis of Oligonucleotide” described in a paragraph 10.42 of Molecular cloning 3^{rd} ed (Sambrook and Rusell, Cold Spring Harbor Laboratory Press, New York, USA, 2001) using a DNA Synthesizer Model 392 from the company Applied biosystems. The synthesized primers were also purified with an OPC (Oligonucleotide Purification Cartridge) column, quantitified using a UV spectrophotometer, dried using a Speed Vac system, and then dissolved in distilled water to a suitable concentration to obtain a primer composition, which was used as the PCR primers.

### Example 3

### Extraction of Mycobacterial DNA from Clinical Specimen

2 to 4 [symbol] of sputum from a suspected tuberculosis patient and an equivalent amount of 4N NaOH were put into a 15 [symbol] tube, sufficiently stirred, and then centrifuged at 4,000 rpm for 20 minutes. Supernatant was removed and 10 [symbol] of a PBS buffer (137 mM NaCl, 2.7 mM KCl, 10 mM Na₂HPO₄, 2 mM KH₂PO₄) was added into the resultant precipitate, and the resultant mixture was sufficiently stirred, and then centrifuged at 4,000 rpm for 20 minutes. Then, supernatant was removed and the resultant precipitate was transferred to a 1.5 [symbol] tube, and 1 [symbol] of PBS buffer was added thereto, stirred, and then centrifuged at 13,000 rpm for 5 minutes. Then, supernatant was removed and 50 to 100 [symbol] of 5% Chelex 100 resin (Bio-Rad) was added to the resultant precipitate, and the resultant mixture was heated at 100° C. for 20 minutes, and then centrifuged at 13,000 rpm for 3 minuted to obtain DNA supernatant, which was used as a template DNA in a PCR reaction.

### Example 4

### Multiplex One-Tube Nested PCR Using the Primers

PCR Reaction

Total 20 [symbol] of a PCR reaction solution was prepared by putting into a PCR tube 2 [symbol] of a 10X buffer, 1 [symbol] of 2 mM dNTP, 0.5 [symbol] of 1 uM mpb64 primary primer, 1 [symbol] of 10 uM mpb64 secondary primer, 0.5 [symbol] of 2 uM HLA-DR primer, 0.5 [symbol] of an HLA-DR PCR clone and 9.5 [symbol] of distilled water, followed by adding 0.5 [symbol] of 1 units/ [symbol] Taq polymerase and 4.5 [symbol] of DNA extracted in Example 3. PCR reaction was carried out using a PCR system (Perkin Elmer 9600, USA) under the following conditions: one denaturation cycle at 95° C. for 5 minutes; and 15 amplification cycles consisting of a denaturation step at 94° C. for 40 seconds, an annealing step at 66° C. for 40 second and an extension step at 72° C. for 40 second, followed by 35 amplification cycles consisting of a denaturation step at 94° C. for 40 seconds, an annealing step at 56° C. for 40 second and an extension step at 72° C. for 40 second; and one extension cycle at 72° C. for 5 minutes.

### Example 5

### Confirmation of Multiplex One-Tube Nested PCR Product

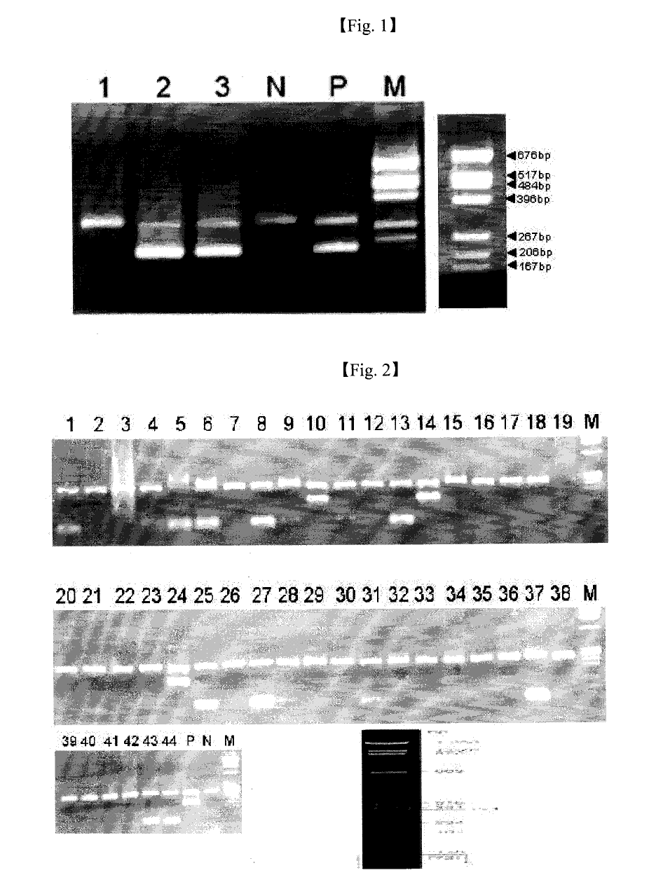

After the multiplex one-tube nested PCR reaction was completed, 1 [symbol] of a gel loading buffer (0.25% bromophenol blue, 0.25% xylene cyanol FF, 15% Ficoll 400) was mixed with 5 [symbol] of the resultant PCT product, and the resultant mixture was electrophoresed at a 100 to 150 volt current for 30 to 60 minutes in a 2% agarose gel containing 1 [symbol] of ethidium bromide (EtBr), and then PCR bands were observed on an image analyzer (Vilber Lourmat, France) provided with a UV transilluminator. At this time, BioCore Marker was used as the standard marker. FIG. 1 is an electrophoretic gel image showing a result of a multiplex one-tube nested PCR method obtained by using a template DNA of the strain separated from the clinical specimen, and the primers of the present invention. In the electrophoretic gel image, Lane 1 represents a result experimenting with a template DNA separated from a clinical specimen of a normal human, and Lanes 2 and 3 represent results experimenting with template DNAs separated from clinical specimens of patients suffering from M. tuberculosis. In this example, an M. tuberculosis reference strain ATCC 27294, kindly provided from American Type Culture Collection(ATCC), was used as the positive control (Lame P), and a Chlamydia trachomatis reference strain ATCC 33530 was used as the negative control (Lane N).

Lane 1 shows a 276-bp single band (HLA-DR gene); Lanes 2 and 3 show two bands having sizes of 276 bp (HLA-DR gene) and 170 bp (a product of the mpb64 gene by the secondary primer); Lane 4 (Lane N) is M. tuberculosis group-negative and shows a 276-bp single band (HLA-DR gene); and Lane 5 is M. tuberculosis group-positive and shows two bands having sizes of 276 bp (HLA-DR gene) and 170 bp (a product of the mpb64 gene by the secondary primer).

Accordingly, the Lane 1 was proven to be M. tuberculosis group-negative and the Lanes 2 and 3 were proven to be M. tuberculosis group-positive.

### Example 6

### Diagnostic Effect of a Conventionally Used Kit (IS6110) and a Kit of the Present Invention on M. Tuberculosis

A conventional commercially available IS6110 kit (Biocore, Republic of Korea) and the kit of the present invention were used to compare the detection effects on the strain M. tuberculosis.

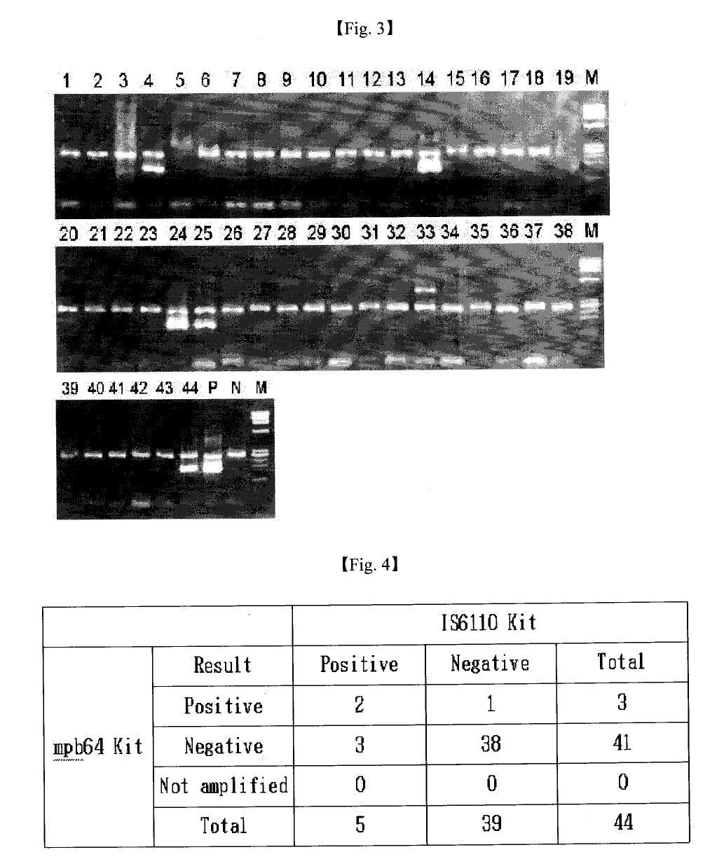

For the IS6110 kit, Mycobacterial DNA was obtained in the same manner as in the method for extracting Mycobacterial DNA from the clinical specimen of the present invention (see Example 3). Total 20 [symbol] of a PCR reaction solution was prepared by putting into a PCR tube 2 [symbol] of a 10X buffer, 1 [symbol] of 2 mM dNTP, 0.5 [symbol] of luM mpb64 primary primer, 1 [symbol] of 10 uM mpb64 secondary primer, 0.5 0 [symbol] of 2 uM HLA-DR primer, 0.5 [symbol] of an HLA-DR PCR clone, 0.3 [symbol] of cresol red and 9.2 [symbol] of distilled water, followed by adding 0.5 [symbol] of 1 units/ [symbol] Taq polymerase and 4.5 [symbol] of DNA extracted in Example 3. PCR reaction was carried out using a PCR system (Perkin Elmer 9600, USA) under the following conditions: one denaturation cycle at 95° C. for 5 minutes; and 15 amplification cycles consisting of a denaturation step at 94° C. for 40 seconds, an annealing step at 66° C. for 40 second and an extension step at 72° C. for 40 second, followed by 35 amplification cycles consisting of a denaturation step at 94° C. for 40 seconds, an annealing step at 56° C. for 40 second and an extension step at 72° C. for 40 second; and one extension cycle at 72° C. for 5 minutes. The PCR condition described above was identical to that of the present invention using the mpb64 gene, except that the different primers were used owing to difference of the PCR target gene. After PCR amplification of the IS6110 kit, an experiment was repeated in the same manner as described in Example 5. It is proven to be M. tuberculosis group-positive if a 181 -bp PCR product (a product of the IS6110 gene by the secondary primer) is amplified, and M. tuberculosis group-negative if only the 276-bp HLA-DR gene is amplified. The results are shown in FIGS. 2 to 4.

As shown in FIGS. 2 to 4, it was proven that two Samples 14 and 24 were positive in the IS6110 kit and negative in the mpb64 kit, and three Samples 4, 25 and 44 were positive only in the IS6110 kit. On the contrary, it was proven that Sample 10 was positive only in the mpb64 PCR kit. It was proven that only two Samples 14 and 24 were truly positive when these samples were analyzed using another method. As described above, it was judged that the kit of the present invention had the excellent accuracy when compared to the conventional IS6110 kit since the conventional IS6110 PCR kit had the higher false-positive rate than the mpb64 kit of the present invention.

### INDUSTRIAL APPLICABILITY

As collectively described above, according to the three pairs of the primers of the present invention and the multiplex one-tube nested PCR method using the same, the M. tuberculosis and non-M. tuberculosis groups can be effectively detected in the specimens with the excellent sensitivity and specificity within a shorter time, compared to the conventional methods for diagnosing the M. tuberculosis group. Also, the method of the present invention can improve reliability of the experimental results since it may judge the false-negative possibility by reaction inhibitors, which may be present in the DNA solution extracted from the clinical specimens.

1. A primer composition for detecting Mycobacterium sp., comprising at least two primers selected from the group consisting of primers set forth in SEQ ID NO: 1 to SEQ ID NO: 6.

2. The primer composition for detecting Mycobacterium sp. according to claim 1, wherein the composition comprises at least one primer selected from the group consisting of SEQ ID NO: 1, SEQ ID NO: 2, SEQ ID NO: 3 and SEQ ID NO: 4, and a primer selected from the group consisting of SEQ ID NO: 5 and SEQ ID NO: 6.

3. A kit for detecting Mycobacterium sp., comprising a container comprising the primer composition as defined in claim 1.

4. A method for detecting Mycobacterium sp., comprising:
a) extracting DNA from a sample;
b) mixing the primer composition as defined in claim 1 with the extracted DNA in the same container;
c) PCR-amplifying the DNA in the primer mixture; and
d) verifying the amplified PCR product.

5. The method for detecting Mycobacterium sp. according to claim 4, wherein the sample is selected from the group consisting of blood, sputum, saliva, urine, cerebrospinal fluid, pleural fluids, pleural biopsies or bronchial washing fluid.

6. The kit for detecting Mycobacterium sp. according to claim 3, wherein the primer composition comprises at least one primer selected from the group consisting of SEQ ID NO: 1, SEQ ID NO: 2, SEQ ID NO: 3 and SEQ ID NO: 4, and a primer selected from the group consisting of SEQ ID NO: 5 and SEQ ID NO: 6.

7. The method for detecting Mycobacterium sp. according to claim 4, wherein the primer composition comprises at least one primer selected from the group consisting of SEQ ID NO: 1, SEQ ID NO: 2, SEQ ID NO: 3 and SEQ ID NO: 4, and a primer selected from the group consisting of SEQ ID NO: 5 and SEQ ID NO: 6.


SEQUENCE LISTING (6 sequences)

SEQ ID NO: 1 - DNA, 26 bp, Artificial Sequence
  Description of Artificial Sequence Synthetic primer
cccagttactaccccgaccagaagtc

SEQ ID NO: 2 - DNA, 29 bp, Artificial Sequence
  Description of Artificial Sequence Synthetic primer
ccacagcgtgtcataggtgattggcttgc

SEQ ID NO: 3 - DNA, 21 bp, Artificial Sequence
  Description of Artificial Sequence Synthetic primer
agtcgctggaaaattacatcg

SEQ ID NO: 4 - DNA, 18 bp, Artificial Sequence
  Description of Artificial Sequence Synthetic primer
ggcgttctggtagacctt

SEQ ID NO: 5 - DNA, 20 bp, Artificial Sequence
  Description of Artificial Sequence Synthetic primer
tccccacagcacgtttcttg

SEQ ID NO: 6 - DNA, 20 bp, Artificial Sequence
  Description of Artificial Sequence Synthetic primer
ccgctgcactgtgaagctct